(19) United States
(12) Patent Application Publication
(10) Pub. No.: US 2025/0255601 A1
(43) Pub. Date: Aug. 14, 2025

(54) A SYSTEM FOR EXTRACORPOREAL AND INTRACORPOREAL WOUND CLOSURE AND A SUTURE CARTRIDGE THEREOF

(71) Applicant: DENOVO BIOINNOVATIONS PRIVATE LIMITED, Maharashtra (IN)

(72) Inventors: Nikhil MAMORIA, Maharashtra (IN); Himanshu LADDHA, Maharashtra (IN); Rajendra Narayan LENKA, Odisha (IN)

(21) Appl. No.: 18/857,642

(22) PCT Filed: Apr. 18, 2023
(86) PCT No.: PCT/IB2023/053962

§ 371 Date: Oct. 17, 2024

(30) Foreign Application Priority Data
Apr. 18, 2022 (IN) 202221022786

(51) Int. Cl. A61B 17/04; A61B 17/00; A61B 17/06; A61B 34/30

(52) U.S. Cl. CPC A61B 17/0469; A61B 17/0482; A61B 17/0483; A61B 17/0491; A61B 17/06066; A61B 17/06114; A61B 34/30; A61B 2017/00473; A61B 2017/06142

(57) ABSTRACT

A system for performing any of extracorporeal or intracorporeal wound closure, includes a suture cartridge 100 configured to be detachably coupled to a distal end of a handle of the system extracorporeal wound closure, or to a shaft assembly that is coupled to distal end of the handle for intracorporeal wound closure. On being coupled to any of the shaft assembly or the handle, a driving pulley 112A of the cartridge 100 gets operatively coupled to a driving mechanism in the handle to drive a belt 110 of the suture cartridge 100 to drive a suturing needle 106 for suturing tissues for the extracorporeal or intracorporeal wound closure. The cartridge 100 can be coupled with any of a robotic system, a hand held system and a minimal invasive surgical system.

### TECHNICAL FIELD

The present disclosure relates to the field of surgical instruments. In particular, the present disclosure relates to a suture cartridge containing a curved needle with thread and needle-driving assembly, which can be used for all types of wound closures during extracorporeal and intracorporeal suturing.

### BACKGROUND

Background description includes information that may be useful in understanding the present invention. It is not an admission that any of the information provided herein is prior art or relevant to the presently claimed invention, or that any publication specifically or implicitly referenced is prior art.

Suturing is the most ancient and most effective method of wound closure. In suturing, the two edges of the wound are approximated using suture material through a series of knots. Suturing is the most commonly performed method of wound closure in all types of surgeries including open surgeries, minimally invasive surgeries like laparoscopy, thoracoscopy, arthroscopy, robotic surgeries, and endoscopic surgeries. Suturing is also performed in Natural Orifice transluminal Endoscopic surgeries

Predominantly, the surgeon has to perform suturing manually with the help of a needle holder, grasper, and curved suturing needle with suture thread. The curved suture needle has one sharpened end meant for passing through the tissues or taking a bite for suturing, whereas the opposite end holds the suture material. There are mainly 2 types of suturing continuous and interrupted sutures. Continuous suture-running stitches, continuous sutures are a series of stitches taken with one strand of thread material. The strand may be tied to itself at each end, or looped, with both cut ends of the strand tied. Interrupted suture-Interrupted sutures use a number of strands to close the wound. Each strand of thread is tied and cut after insertion. This provides a more secure closure, because if one suture breaks, the remaining sutures will hold the wound edges in approximation

This entire process of stitching consists of: orienting the needle, positioning the needle accurately, driving the needle with force, and grabbing the needle on the exit of tissue, followed by tying the knot taking loops on graspers, and taking the loose end of suture material through these loops make a knot, this is how one suture is placed. And these steps are repeated multiple times till the whole wound is closed.

Ideal suturing IS defined as advancing a needle along its curvature (needle circle) to minimize tissue trauma while placing the suture with its intended span and tissue bite in the expected place. Technically, for near-ideal suturing, the needle holder should position and grasp the needle about ⅔ of the way back from the tip. Then orient the needle to the surface of the tissue and place the tip of the needle at this position so that the sharp tip enters the tissue at right angles. When approximately ½ of the curve of the needle has entered the tissue the wrist is supinated and lifted slightly to pass the curved section of the needle through the tissue. The emerging tip of the needle is then grasped using a grasper in the left hand. The tip of the needle should penetrate the surface perpendicularly and be rotated all the way by supinating the right hand to rotate the needle through its arc. Repeat the same motion of the needle for penetration on the other edge of the tissue. Once the suture has passed through the two edges, the thread is pulled through leaving the tail of the suture behind. Then the needle is handed from the grasper to the needle holder for further steps of knotting. This requires dual-hand coordination and is a cumbersome task. If not held or aligned properly, the surgeon has to drop the needle, pick it up again using his/her own judgment, and then try to stitch together the departed tissues. This not only becomes time-consuming, but the wrong alignment of the needle may inadvertently injure the otherwise healthy tissues or poor anastomosis. Due to this range of movement, the surgeon's hand often trembles while suturing. This becomes a significantly bigger problem considering the delicate and crucial nature of the suturing.

The points between the entry and exit of the needle should be equal and equidistant. The exact span of the suturing points, both exit and entry are determined by the span of the needle used and the direction of the force on the needle.

There is a large solution gap with the conventional needle holders used during intracorporeal or extracorporeal suturing. First of such solution gaps relates to the needle positioning and orientation. Ability to maneuver the needle into the desired position in the needle holder jaw is one of the first skills acquired by a laparoscopic surgeon.

Further, during the suturing, the surgeon needs to constantly change the orientation of the needle in order to find the appropriate pose. To this end, the reorientation phase is conducted through successive grasp and release movements.

Still further, the needle must next be positioned correctly in the jaws of the needle holder. Ideally, the needle is grasped in the right orientation by the tips of the jaws at ⅔rd from the tip, and should be oriented such that the directions of (1) the needle tip and (2) the needle-driving force are identical—an optimal direction for the both being 90°, head-on against tissue resistance.

It is a mistake to grasp the needle by the back of the jaws as this impairs the precision needle driving through the tissues and also reduces the grasping force so that the needle swivel is more likely.

Due to the structure of standard needle drivers, the orientation of the needle during the suturing procedure is not completely controllable, and multiple pairs of handoff movements are required to position the needle, before the execution of each Stitch.

An error in suturing can produce significant tissue damage and is more likely to happen when the needle orientation is not completely under the surgeon's control. The successive steps to orient the needle in the desired position are tedious and exhaustive

Next of the solution gaps lies in needle control. Presently, the needle control is accomplished by correctly directing the needle tip as well as the needle-driving force. The needle follows the tip in passing through tissue layers with the least amount of trauma and effort if the tissue resistance, needle tip, grasping point, and direction of force are assembled on the same axis. Therefore, directions of (1) the needle tip and (2) the needle-driving force must be identical; the optimal direction for both is 90°, head-on against tissue resistance. If these directions are dissimilar, the needle will be deflected within the instrument jaws and needle control will be lost.

Conventionally pronation and supination of the hand produce pure rotation about the long axis of the instrument because the hand and forearm are essentially a direct continuation of the instrument shaft.

For the surgeon to follow the arc of the needle through tissue, a movement of revolution is required. In Minimally Invasive Surgeries like laparoscopy, the length of the laparoscopic needle driver results in the transmission of reduced force from the surgeon's hands to the instrument jaws, and consequently diminished needle control as well. To compensate, surgeons seek instruments whose jaws are more powerful. Without awareness of these factors, the application of increased force can easily result in tissue damage or a poorly constructed suture line. In addition, instruments designed to hold the needle more forcefully tend to have a more cumbersome lock mechanism whose deployment can also play a role in increased tissue trauma. If the surgeon attempts to suture laparoscopically, which is probably the most difficult skill to master in the MIS environment, by simply rotating the shaft of the needle holder (i.e. without any revolving movement), the path the needle takes will not be its natural arc. Instead, the needle tip catches only a small bite of tissue and pulls up and away from the tissue, tearing it. A coordinated combination of rotation of the needle holder tip and revolution about the axis formed by the fulcrum action of the trocar is required to achieve the correct entrance angle, obtain sufficient tissue with the needle, and revolve the needle about its imaginary axis. The direction and magnitude of the needle motion than are equal to any given hand movement the surgeon will need to execute smaller, more precise hand movements because any given movement outside the body will translate into a larger motion inside it.

The greater incidence of suturing error can be partly attributed to the ergonomic challenges of Minimally Invasive surgeries. The two-dimensional monoscopic laparoscopic camera systems can cause eye strain; and manipulation of long instruments at awkward angles leads to hand, wrist, and shoulder injuries] A major restriction on maneuverability is the four degrees-of-freedom of movement provided by laparoscopic instruments with the fulcrum effect, where movements of the handle of the instrument in one direction is translated to a movement at the tip of instrument in the opposite direction.

Further challenge is to avoid needle injury during suturing. Needle stick injury (NSI) is defined as percutaneous exposure where the skin is breached by a needle or any sharp object contaminated by blood or other bodily fluid due to accidental pricks. NSIs are one of the dreaded but preventable occupational hazards posed to healthcare workers in various clinical settings. Suture needles are the most common cause of NSIs in the surgical setting and most commonly occur to the surgeon while the suture is being used. Many suture needle injuries occur during the loading or adjustment of the needle in the needle driver. Another cause is the needle manipulation inside the tissue and suture needle disposal.

Therefore, there is a requirement for an improved laparoscopic surgical device that can facilitate suturing by accurate grasping and driving the needle, during laparoscopic surgery overcoming the limitations of conventional laparoscopic surgical devices.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### OBJECTS OF THE INVENTION

A general object of the present disclosure is to overcome the limitations of the conventional suturing method using needle holders, graspers, and suture needles with thread

An objective of the disclosure is to provide a suture cartridge with a loaded suture needle and thread.

Another objective of the disclosure is to provide a customized suture needle for wound closure.

Another objective of the disclosure is to provide a needle-driving mechanism to drive the suture needle.

Another objective of the disclosure is to provide a suture cartridge that avoids needle stick injuries.

Another objective of the disclosure is to provide a suture cartridge that can be used for both types of suturing-continuous and interrupted suturing.

Another objective of the disclosure is to provide a suture cartridge with a needle track such that the needle is ideally oriented and in control.

Another objective of the disclosure is to provide a needle driving mechanism which drives the needle with constant force.

Another objective of the disclosure is to provide the suture cartridge that can be used for all types of wound closure requirements intracorporeal or extracorporeal.

Another objective of the disclosure is to provide a suture cartridge for a surgical instrument for open surgeries.

Another objective of the disclosure is to provide a suture cartridge that can also be used with a laparoscopic instrument for minimally invasive surgeries.

Another objective of the disclosure is to provide a suture cartridge that can also be used for surgical robotics.

Another objective of the disclosure is to provide a suture cartridge for endoscopic surgical instruments

A needle track that will position and orient the needle at the desired angle for accurate entry and exit of needle during suture path and reduce the efforts and time

Another objective of the disclosure is to provide a mechanism for motion transfer from the handle of the surgical instrument to the cartridge using various embodiments of rotary shafts which include flexible and solid shafts

The other objects and advantages of the present invention will be apparent from the following description when read in conjunction with the accompanying drawings, which are incorporated for illustration of the preferred embodiments of the present invention and are not intended to limit the scope thereof.

### SUMMARY

Aspects of the present disclosure relate to a system for wound closure that can be used for any of an intracorporeal or an extracorporeal wound closure. In particular, the disclosed system is based on a suture cartridge that can be detachably coupled to different devices used in surgical procedures, such as a handle of a surgical system for extracorporeal wound closure, or to a shaft assembly of a minimum invasive surgical system for intracorporeal wound closure or a robotic surgical system

In an aspect, the system for any of an extracorporeal or an intracorporeal wound closure includes: a handle; a shaft assembly configured to be detachably coupled to a distal end of the handle; and a suture cartridge configured to be detachably coupled to a distal end of the shaft assembly or the distal end of the handle.

In an aspect, coupling of a proximal end of the shaft assembly to the distal end of the handle and coupling of the suture cartridge to the distal end of the shaft assembly enables the system to be used for intracorporeal wound closure; and wherein coupling of the suture cartridge directly to the distal end of the handle enables the system to be used for extracorporeal wound closure.

In an embodiment, the shaft assembly comprises an outer casing and a flexible shaft housed within the outer casing such that when the shaft assembly is coupled between the handle and the suture cartridge, a proximal end of the flexible shaft is coupled to driving mechanism in the handle and a distal end of the flexible shaft is operatively coupled to the belt for driving the needle.

In an embodiment, a proximal end of the suture cartridge comprises a circular cross section with a plurality of lugs circumferentially arranged on an outer surface of the circular cross section for engagement with corresponding features, such as groves, provided on an inner surface of the shaft assembly or on an inner surface of an opening at the distal end of the handle for detachable coupling of the suture cartridge with any of the distal end of the shaft assembly and the distal end of the handle.

In an embodiment, the proximal end of the outer casing of the shaft assembly comprises a plurality of circumferentially arranged lugs that are similar to the a plurality of circumferentially arranged lugs on the proximal end of the suture cartridge such that it is possible to detachably couple any of the suture cartridge or the proximal end of the shaft assembly to the distal end of the handle.

In an embodiment, the shaft assembly comprises an articulation joint coupled to the outer casing at the distal end of the shaft assembly to enable pivotally moving the suture cartridge about two mutually perpendicular axes, each perpendicular to the longitudinal axis of the suture cartridge, thereby providing rotational freedom to the suture cartridge during wound closure.

In an embodiment, the corresponding groves for engaging with the plurality of lugs of the suture cartridge are provided on an inner surface of a yoke of the articulation joint.

In an embodiment, the handle comprises controls for actuating the driving mechanism and for manipulating the articulation joint.

In an embodiment, the a flexible shaft comprises two or more shafts coupled to each other through any of one or more of springs and articulation joints to provide flexibility to the flexible shaft.

In an embodiment, the a flexible shaft comprises a pair of couplers and a pair of coupling shafts, each coupling shaft coupled to the two or more shafts at the proximal and the distal ends of the flexible shaft through the respective couplers, and wherein the coupling shafts are configured to operatively couple with the driving shaft of the suture cartridge and an output shaft of the driving mechanism, when the shaft assembly is coupled between the handle and the suture cartridge.

In an embodiment, the system for wound closure is any of a robotic system, a hand held system and a minimal invasive surgical system.

Another aspect of the present disclosure relates to a suture cartridge for extracorporeal and intracorporeal wound closure. The suture cartridge is configured to be detachably coupled to a distal end of a handle of a system for wound closure or to a distal end of a shaft assembly that is coupled to the distal end of the handle of the system for wound closure. In an aspect, on the suture cartridge being coupled to any of the shaft assembly or the handle of the system for wound closure, a driving pulley of the suture cartridge gets operatively coupled to a driving mechanism in the handle to enable the driving mechanism to drive a belt of the suture cartridge and thereby to drive a suturing needle for suturing tissues for the extracorporeal or intracorporeal wound closure.

In an aspect, coupling of the suture cartridge to the handle through the shaft assembly enables intracorporeal wound closure; and coupling of the suture cartridge directly to the distal end of the handle enables extracorporeal wound closure.

In an embodiment, the suture cartridge comprises: a needle track configured on an outer periphery of a raised projection with the suturing needle positioned in the needle track for movement along the needle track; a belt configured around the outer periphery of the raised projection such that the belt presses against the suturing needle to move the suturing needle along the needle track as the belt moves; a pair of idler pulleys to route the belt around the outer periphery of the raised projection; and a driving pulley to drive the belt.

In an embodiment, when the suture cartridge is coupled to the distal end of the shaft assembly or the handle, the driving pulley gets operatively coupled to a driving mechanism in the handle, either through the shaft assembly, or directly, to enable the driving mechanism for driving the belt and thereby to drive the suturing needle.

In an embodiment, the suture cartridge comprises a base plate and a cover, wherein the raised projection is configured on a distal side of the base plate, and the pair of idler pulleys and the driving pulley are fixed to the base plate.

In an embodiment, the suture cartridge comprises a driven bevel gear fixedly coupled to the driving pulley, and a driving bevel gear in mesh with the driven bevel gear; and wherein the driving bevel gear is fixed to a distal end of a driven shaft that is configured along a longitudinal axis of the suture cartridge such that when the suture cartridge is coupled to the distal end of the shaft assembly, or the handle, a proximal end of the driven shaft is coupled to a distal end of a flexible shaft of the shaft assembly or directly to the driving mechanism for transferring rotational motion to the belt.

In an embodiment, the driven shaft is rotatably fixed in the longitudinal direction at the proximal end of the base plate such that the driving gear fixed to the driven shaft is in mesh with the driven bevel gear.

In an embodiment, the proximal end of the base plate includes a circular cross section with a plurality of lugs circumferentially arranged on an outer surface of the circular cross section for engagement with corresponding groves provided on an inner surface of an outer casing of the shaft assembly or on an inner surface of an opening at the distal end of the handle for detachable coupling of the suture cartridge with any of the distal end of the shaft assembly and the distal end of the handle.

In an embodiment, the needle track is shaped as a circular arc of more than half circle.

In an embodiment, the suture needle is a circular needle with an arc angle of more than 200 degrees.

In an embodiment, the belt comprises a grove on an outer surface of the belt, the grove being of semi-circular cross section to accommodate the suture needle and simultaneously press the suture needle for movement of the suture needle in the needle track.

In an embodiment, the suture cartridge is configured for use with any of a robotic surgery system, a hand held surgical system and a minimal invasive surgical system.

Yet another aspect of the present disclosure relates to a suture head for wound closure that includes a needle track with the suturing needle positioned in the needle track for movement along the needle track; a belt configured around the outer periphery of the needle track such that the belt presses against the suturing needle to move the suturing needle along the needle track as the belt moves; a pair of idler pulleys to route the belt around the needle track; and a driving pulley to drive the belt. In an aspect, operatively coupling the suture head to a driving mechanism of a wound closure device/surgical apparatus enables driving the belt and thereby the suturing needle for suturing.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 illustrates an exemplary exploded view of the disclosed suture cartridge, in accordance with an embodiment of the present disclosure.

FIGS. 2A to 2D illustrate different exemplary views of a base plate of the suture cartridge of FIG. 1, in accordance with an embodiment of the present disclosure.

FIGS. 3A to 3E illustrate different exemplary views of an outer cover of the suture cartridge of FIG. 1, in accordance with an embodiment of the present disclosure.

FIGS. 4A and 4B illustrate exemplary views of a suture needle of the suture cartridge of FIG. 1, in accordance with embodiments of the present disclosure.

FIGS. 5A to 5C illustrate different exemplary views a driving pulley and driven bevel gear assembly of the suture cartridge of FIG. 1, in accordance with embodiments of the present disclosure.

FIGS. 6A to 6C illustrate different exemplary views of a belt of the suture cartridge of FIG. 1, in accordance with an embodiment of the present disclosure.

FIGS. 7A and 7B illustrate exemplary perspective view and exploded view respectively of a flexible shaft of a system for wound closure, in accordance with a first embodiment of the flexible shaft.

FIGS. 8A and 8B illustrate exemplary perspective view and exploded view respectively of a flexible shaft of a system for wound closure, in accordance with a second embodiment of the flexible shaft.

FIGS. 9A and 9B illustrate exemplary perspective view and exploded view respectively of a flexible shaft of a system for wound closure, in accordance with a third embodiment of the flexible shaft.

FIGS. 10A and 10B illustrate exemplary perspective views of a yoke of an articulation joint of the shaft assembly and a proximal end of the suture cartridge respectively, each having features for detachable coupling of the suture cartridge with the shaft assembly, in accordance with a third embodiment of the flexible shaft.

FIG. 11 illustrates an exemplary perspective view of a system for wound closure having a suturing head, a shaft assembly and a handle for intracorporeal wound closure, in accordance with an embodiment of the present disclosure.

FIGS. 12A and 12B illustrate exemplary views of the suture cartridge for coupling to a hand-held device for wound closure, in accordance with an embodiment of the present disclosure.

FIGS. 13A and 13B illustrate exemplary views of the suture cartridge for coupling to a robotic surgical system for wound closure, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Various terms are used herein. To the extent a term used in a claim is not defined, it should be given the broadest definition persons in the pertinent art have given that term as reflected in printed publications and issued patents at the time of filing.

Embodiments described herein relate to a wound closure system for use during surgical procedures. The proposed wound closure system is based on a suture cartridge that can be detachably operatively coupled to any of a hand held surgical device, a minimal invasive surgical device/system and a robotic surgical system, thereby allowing any of an intracorporeal and extracorporeal suturing and wound closure. Thus, the suture cartridge can be integrated with various types of embodiments and devices for different types of surgeries like open surgery, minimally invasive surgeries, and robotic surgery. The cartridge may be integrated with various embodiments for intracorporeal suturing in minimally invasive surgeries, such as laparoscopy, thoracoscopy, endoluminal endoscopy, perivisceral endoscopy and arthroscopy, and extracorporeal suturing.

In an aspect, the disclosed suture cartridge includes a curved suture needle and needle-driving mechanism to assist a surgeon in efficiently suturing and knotting tissue as a prelude to a surgical operation during wound closure. The suture cartridge reproduces the same technical phases of traditional suturing technique in a choreographed manner mimicking conventional suturing, thus choreographing the whole suturing process into a precise set of actions. Each maneuver is designed to help make the suturing process smooth and reproducible with the economy of movement and structured choreography so that suturing is efficient with minimum effort, reduced error, and wastage of time.

The suturing precision is deployed by enabling surgeons to reach the desired angle, control bites, secure knots and tighten the knot, as well as to have maximum control of the needle during suturing and knot tying, which may reduce the risk of needle loss

The disclosed suture cartridge consists of a needle grasping and driving mechanism incorporating a more than 200-degree circle curved needle moving along its arc in a designed needle track such that it passes through the tissue at the correct angle and depth for the suturing to take effect without any difficult hand maneuvers, mimicking the steps as is done in surgery to achieve the intended results.

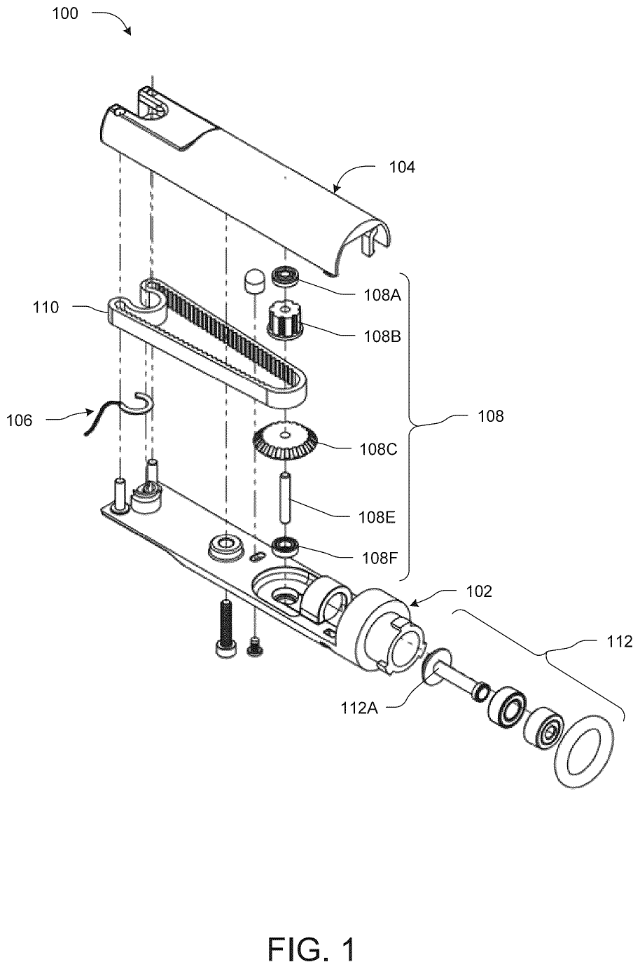

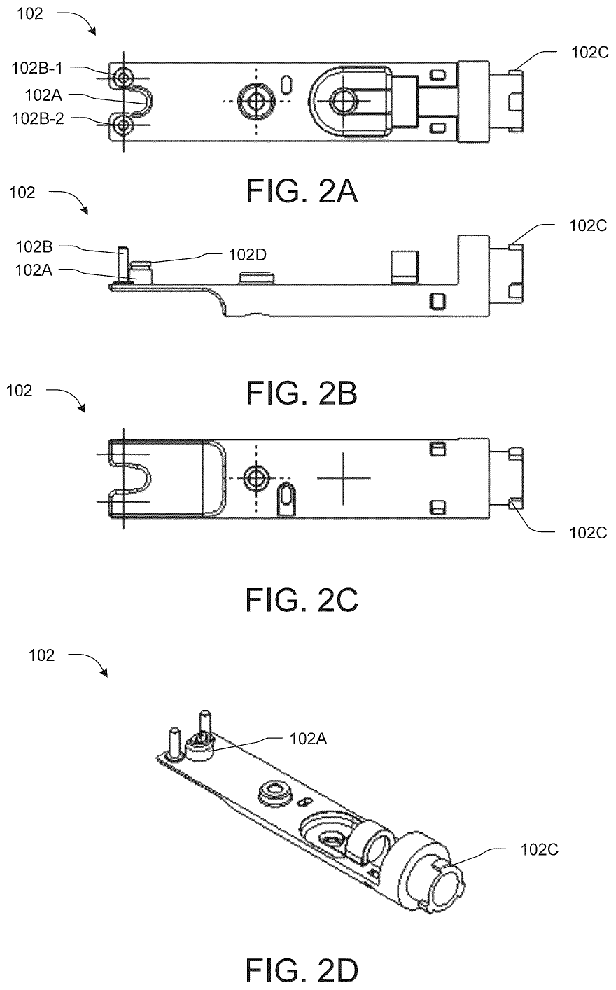

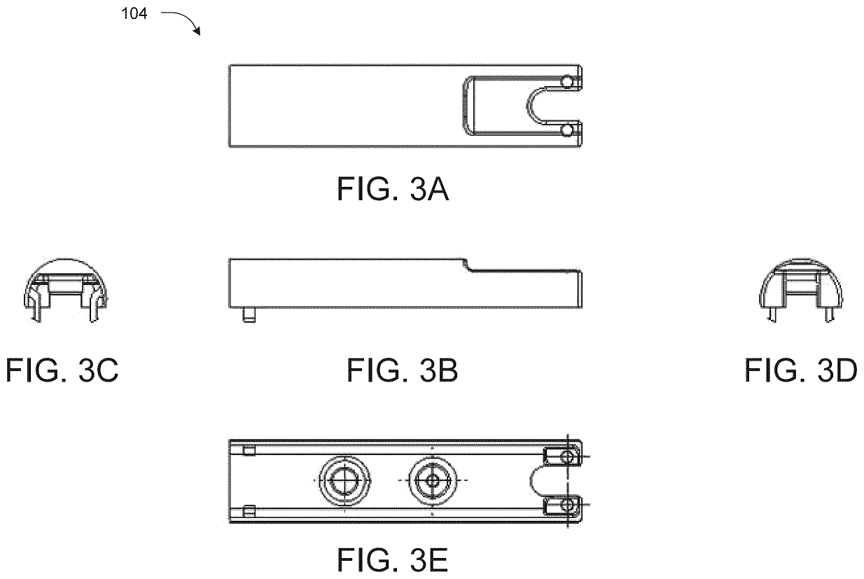

Referring to FIGS. 1 to 6C, where an exploded view of the suture cartridge and different components thereof are disclosed, the suture cartridge 100 can include a base plate 102 and a cover 104. The base plate 102 can include, at a distal side of the base plate 102, a raised projection 102A (refer to FIG. 2B) that includes a needle track 102D (refer FIG. 2B) configured on an outer periphery of the raised projection 102A. A suturing needle 106 with suture is positioned in the needle track 102 D for movement along the needle track 102D.

The suture cartridge 100 further includes a belt 110 configured around the outer periphery of the raised projection 102A such that the belt 110 presses against the suturing needle 106 to move the suturing needle along the needle track 102D as the belt 110 is driven/moved.

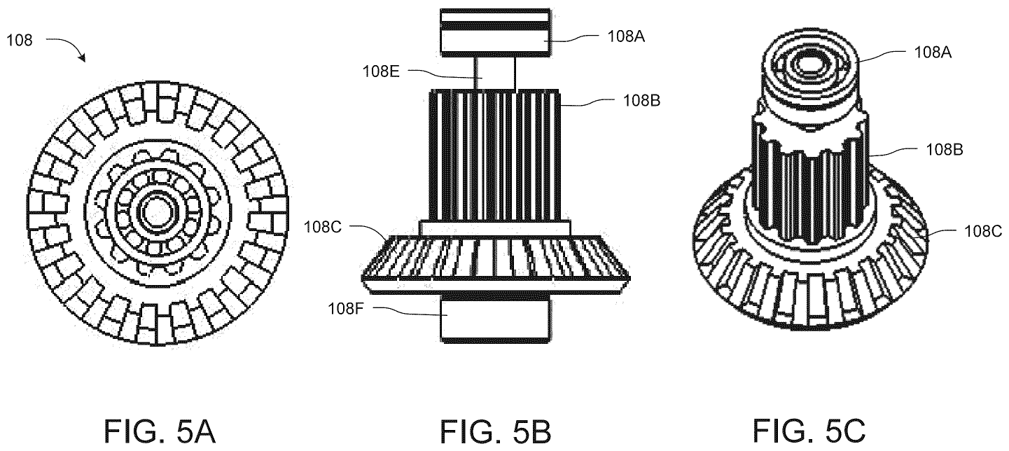

The belt 110 is routed around the outer periphery of the raised projection 102A by means of a pair of idler pulleys 102B. The suture cartridge 100 also includes a driving pulley and driving bevel gear assembly 108 that includes a driving pulley 108B to drive the belt 110. The driving pulley and driving bevel gear assembly 108 includes, besides the driving pulley 108B, a driven bevel gear 108C fixedly coupled to the driving pulley 108B through a shaft 108E. The shaft 108E is supported by bearings 108A and 108F fixed to the cover 104 and the base plate 102 respectively (also refer to FIGS. 5A-5C).

The suture cartridge 100 further includes a driving bevel gear assembly 112 fixed at the base plate 102 at a proximal end of the suture cartridge 100 along a longitudinal axis of the suture cartridge 100. The driving bevel gear assembly 112 includes a driving bevel gear 112A having a driving shaft, and is fixed in the base plate 102 by bearings and washers. The driving bevel gear 112A is configured such that it is in mesh with the driven bevel gear 108C.

The suture cartridge 100 can further include a belt tensioner to ensure an adequate pressure from the belt 110 to the needle 106 for movement of the needle 106 without slipping.

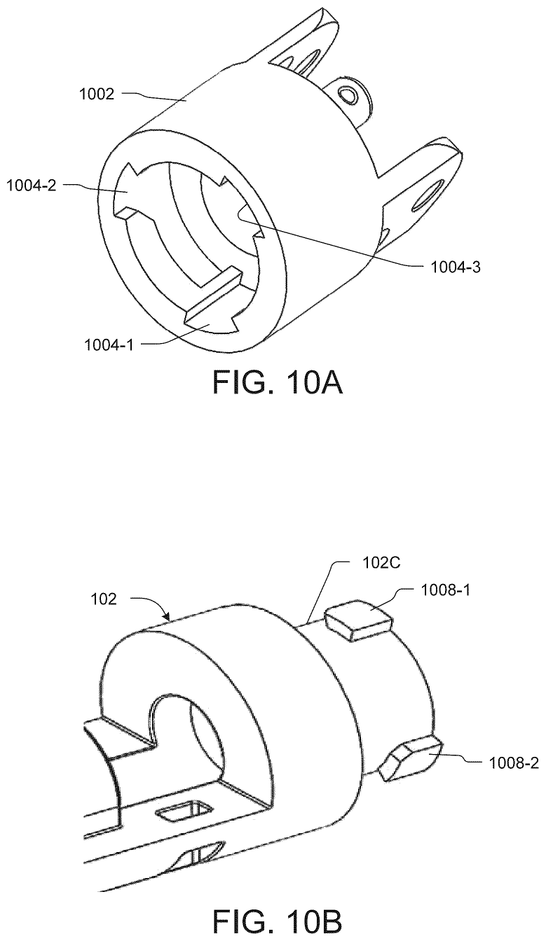

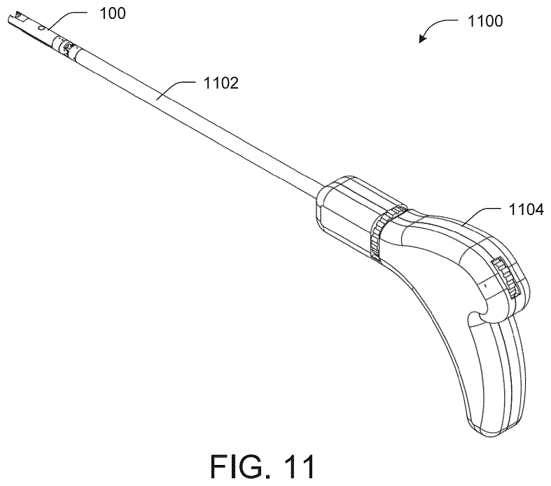

As shown in FIGS. 2A-2D, the base plate 102 includes a circular cross section 102C at a proximal end of the suture cartridge 100. The circular cross section 102C includes a plurality of lugs, such as lugs 1008-1 and 1008-2 (hereinafter lugs 1008), as shown in FIG. 10B. The circular cross section 102C and the lugs 1008 provided on the base plate 102 help detachable coupling of the suture cartridge 100 with different devices of wound closure systems, such as to a handle or a shaft assembly, of the wound closure systems, such as wound closure systems shown in FIGS. 11 to 13B.

In an aspect, when the suture cartridge 100 is coupled to a distal end of a shaft assembly or a handle of a wound closure system, the driving pulley 108B gets operatively coupled to a driving mechanism in the handle, either through the shaft assembly, or directly, to enable the driving mechanism to drive the belt 110 and thereby to drive the suturing needle 106 for suturing operations. Specifically, when the suture cartridge 100 is coupled to the distal end of the shaft assembly, or the handle, a proximal end of the driven shaft is coupled to a distal end of a flexible shaft of the shaft assembly or directly to the driving mechanism in the handle for transferring rotational motion to the belt.

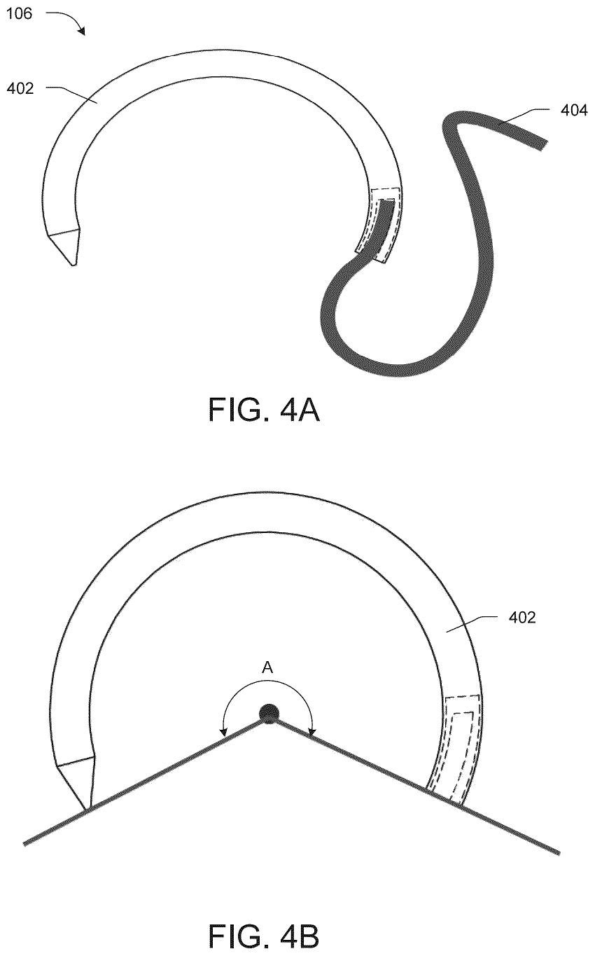

Referring to FIGS. 4A and 4B, as shown, the suture needle 106 includes a circular needle 402 with an arc angle of more than 200 degrees. It further includes a suture/thread 404 fixed at one end. In accordance with the shape of the needle 106, the needle track 102D is also shaped as a circular arc of more than half circle. A needle track 102D of more than half circle prevents the needle 106 from getting dislodged from the needle track 102D, as the needle 106 moves along the needle track 102D. In an aspect, the suture needle 106 can have an arc angle A of more than 200 degrees.

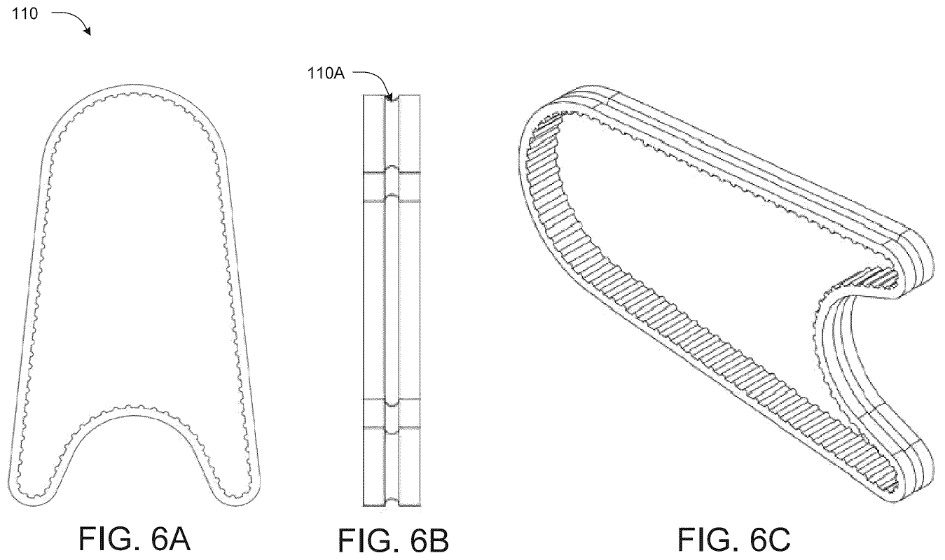

FIGS. 6A-6C show different views of the belt 110. The belt 110 can be a timing belt and can include a grove 110A along a complete length on an outer surface of the belt 110. The grove 110A can be of semi-circular cross section to accommodate the suture needle 106 and simultaneously press the suture needle 106 for the movement of the suture needle 106 in the needle track 102D.

It is to be appreciated that while the configuration of the needle track 108D, the suture needle 106, the needle driving mechanism comprising the belt 110 has been described here with reference to a suture cartridge that includes a detachable feature, the features of needle track 108D, the suture needle 106, the needle driving mechanism comprising the belt 110 can also be used in suturing heads that are not detachable, i.e., have a fixed configuration, with advantageous results in the suturing operations.

FIG. 11 shows an exemplary system 1100 for wound closing having the suture cartridge 100, a handle 1104 and a shaft assembly 1102 that is configured for coupling between the suture cartridge 100 and the handle 1104. In an aspect, the handle 1104, the shaft assembly 1102 and the suture cartridge 100 are configured such that the suture cartridge 100 can be coupled directly to the handle 1104, or through the shaft assembly 1102. When coupled directly to the handle 1104, the set up can be used for extracorporeal suturing, and when coupled to the handle 1104 through the shaft assembly 1102, the set up can be used for intracorporeal suturing.

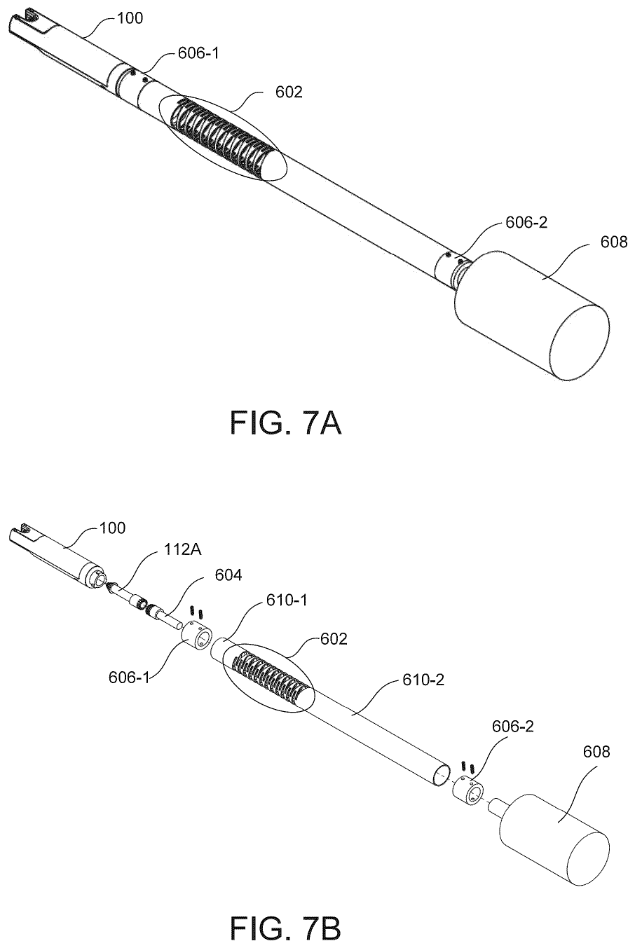

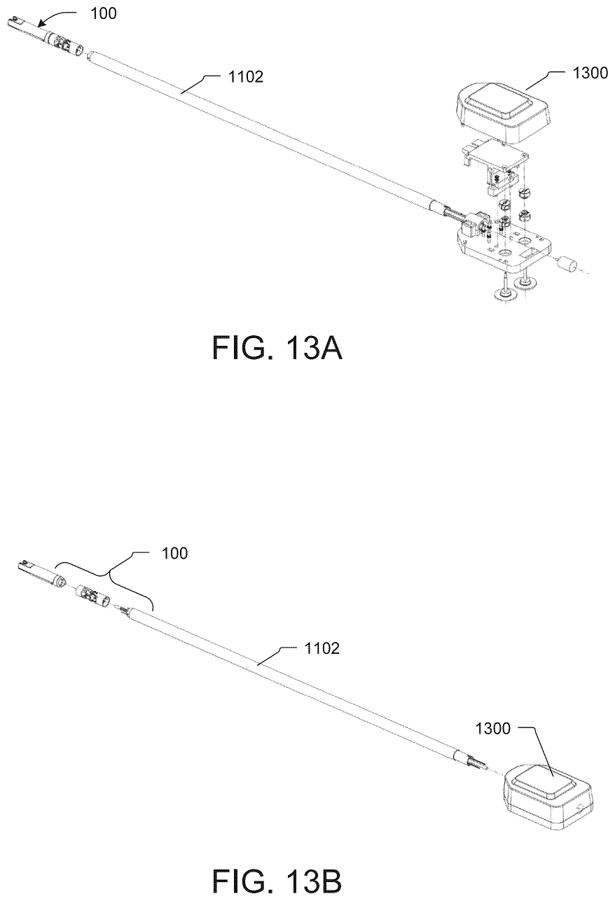

Referring to FIGS. 7A to 9B, showing different configurations of a flexible shaft for shaft assembly 1102 of the wound closing systems, such as shaft assembly 1102 shown in FIG. 11, FIGS. 13A and 13B, the shaft of the shaft assembly 1102 can be an inner part of the shaft assembly 1102 located within an outer casing (also referred to simply as casing) of the shaft assembly 1102. When the shaft assembly 1102 is coupled between a handle, such as handle 1104 shown in FIG. 11, and the suture cartridge 100, the casing is coupled to a housing of the handle 1104 and the base plate 102 of the suture cartridge 100, and the flexible shaft is coupled to a driving mechanism in the handle and to the driving shaft of the driving bevel gear 112A to transfer drive from the driving mechanism to the belt 110.

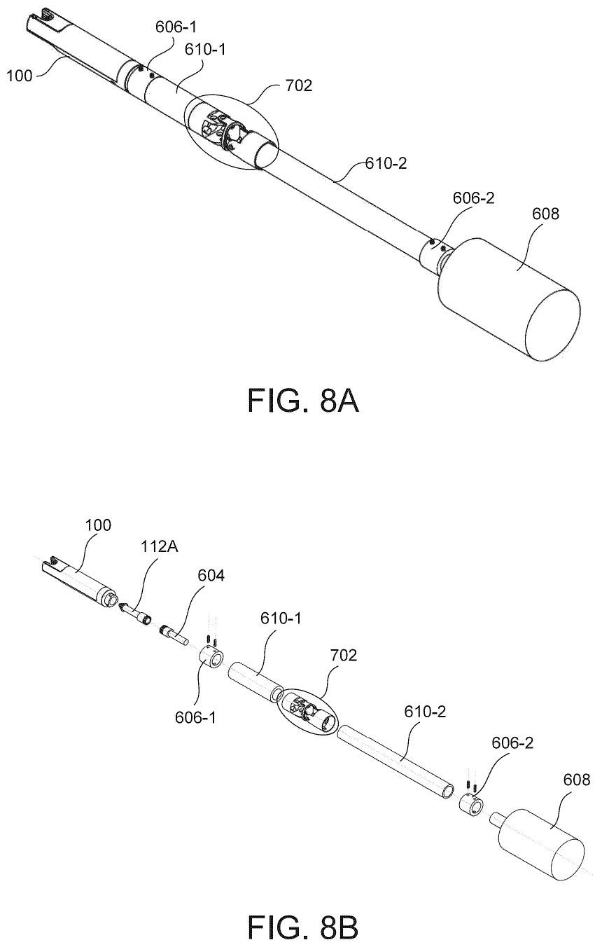

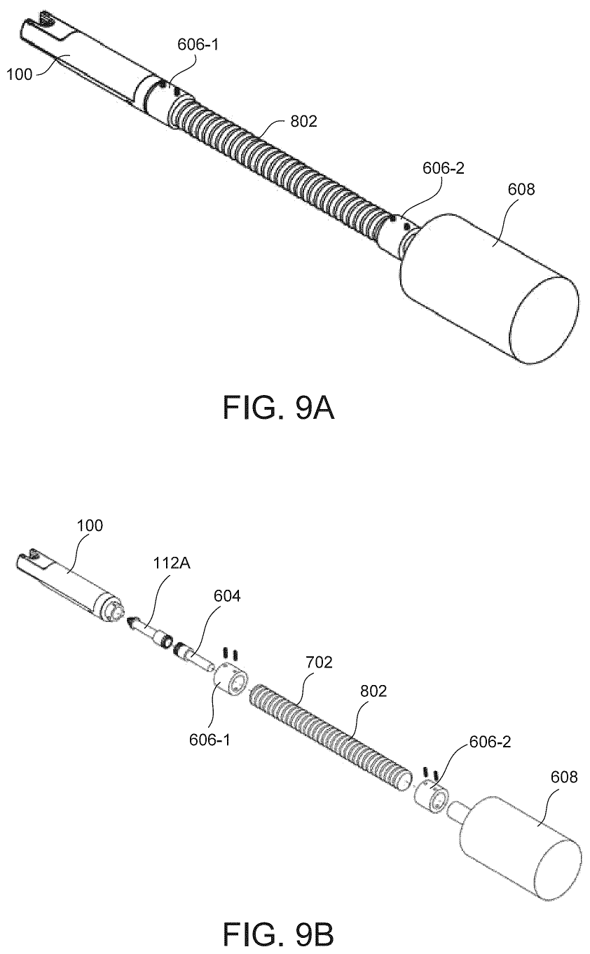

The flexible shaft can be fully flexible, as shown in FIGS. 9A and 9B, along a complete length, or can include two or more shafts such as shafts 610-1 and 610-2, as shown in FIGS. 7A-8B. The shafts 610 can be coupled to each other through one or more of springs 602 as shown in FIGS. 7A and 7B, or through articulation joints 702 as shown in FIGS. 8A and 8B, to provide flexibility to the flexible shaft.

The flexible shaft can include a pair of couplers, such as 606-1 and 606-2 (individually/collectively referred to as coupler/couplers 606, herein) and a pair of coupling shafts 504. Each of the coupling shaft 504 can be coupled to shafts 610 at the proximal and the distal ends of the flexible shaft through the respective couplers 606. When the shaft assembly is coupled between the handle 1104 and the suture cartridge 100, the coupling shafts 504 can be configured to operatively couple with the driving shaft 112A of the suture cartridge 100 and the driving mechanism, such as a motor 608. However, the driving mechanism can be any other mechanism, such as a mechanical mechanism, a manual mechanism and a combination of a motor and a mechanical mechanism, to name a few.

In an embodiment, the shaft assembly 1102 can include an articulation joint coupled to the casing at the distal end of the shaft assembly 1102 to enable pivotally moving the suture cartridge 100 about two mutually perpendicular axes, each perpendicular to the longitudinal axis of the suture cartridge. The articulation joint enables rotational freedom to the suture cartridge during wound closure.

In an embodiment, as shown in FIGS. 10A and 10B, an inner surface of a yoke 1002 of the articulation joint can be configured to receive the circular cross section 102C of the suture cartridge 100 for coupling of the suture cartridge 100 with the distal end of the shaft assembly 1102. The lugs 1008 can engage with groves 1004-1, 1004-2, 1004-3 (collectively referred to as groves 1104, herein) provided on an inner surface of a yoke 1002 of the articulation joint. In an embodiment, the handle 1102 can have controls for actuating the driving mechanism and for manipulating the articulation joint for orienting the suture cartridge in a desired direction.

It is to be appreciated that on an inner surface of an opening at the distal end of the handle 1104 can include a plurality of groves similar to groves 1004 on the yoke 1002, and the proximal end of the outer casing of the shaft assembly 1102 can have a plurality of circumferentially arranged lugs that are similar to the a plurality of circumferentially arranged lugs 1008 on the proximal end of the suture cartridge 100 such that it is possible to detachably couple any of the suture cartridge 100 or the proximal end of the shaft assembly to the distal end of the handle 1104.

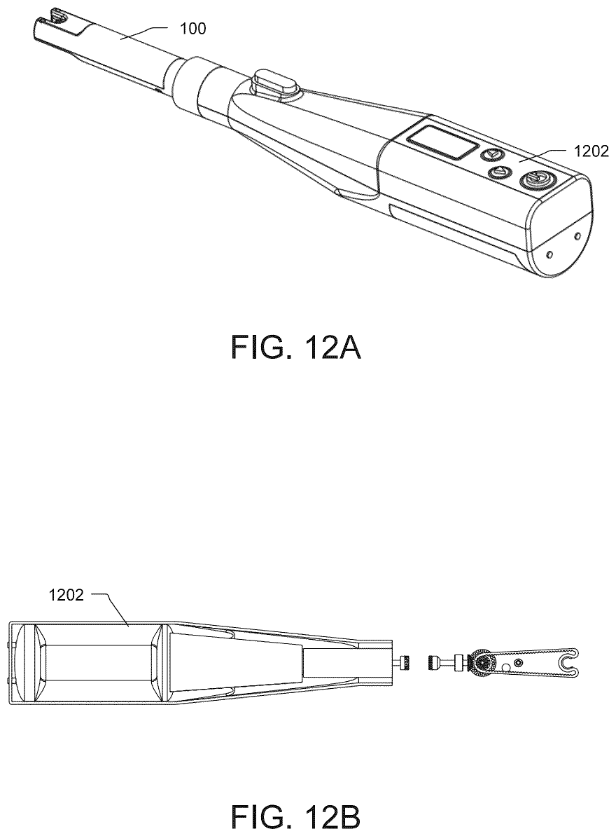

FIGS. 12A and 12B illustrate exemplary views of a suture cartridge 1204 for coupling to a hand-held device 1202 for wound closure, in accordance with an embodiment of the present disclosure.

FIGS. 13A and 13B illustrate exemplary views of the suture cartridge 100 being used with a robotic surgical system 1300 by being coupled to the robotic surgical system 1300 through the shaft assembly 1102. When coupled to the robotic surgical system 1300, the suture cartridge 100 can be used for wound closure.

Thus the present disclosure provides a system for wound closure that can be used for any of an intracorporeal or an extracorporeal wound closure. The disclosed suture cartridge 100 can be detachably coupled to different devices used in surgical procedures, such as a handle of a surgical system for extracorporeal wound closure, or to a shaft assembly of a minimum invasive surgical system for intracorporeal wound closure or a robotic surgical system.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES OF THE INVENTION

The present disclosure provides a suture cartridge based wound closure system that eases suturing for the surgeon.

The present disclosure provides a suture cartridge based wound closure system that helps an adequately experienced/trained person to perform the suturing.

The present disclosure provides a suture cartridge based wound closure system that reduces suturing time, since the surgeon is saved from the process of pronation and supination'

The present disclosure provides a suture cartridge based wound closure system that improves accuracy of suturing near to ideal suturing,

The present disclosure provides a suture cartridge based wound closure system that leads to improved stitches quality resulting in better healing and early recovery.

The present disclosure provides a suture cartridge based wound closure system that avoids NSI during manipulation and disposable.

The present disclosure provides a suture cartridge based wound closure system that reduces number of instruments, such as needle holder, grasper and needle, required for suturing.

1. A system for any of an extracorporeal or an intracorporeal wound closure, the system comprising:
a handle;
a shaft assembly configured to be detachably coupled to a distal end of the handle; and
a suture cartridge configured to be detachably coupled to a distal end of the shaft assembly or the distal end of the handle;
wherein coupling of a proximal end of the shaft assembly to the distal end of the handle, and coupling of the suture cartridge to the distal end of the shaft assembly enables the system to be used for intracorporeal wound closure; and
wherein coupling of the suture cartridge directly to the distal end of the handle enables the system to be used for extracorporeal wound closure.

2. The system as claimed in claim 1, wherein the shaft assembly comprises an outer casing and a flexible shaft housed within the outer casing such that when the shaft assembly is coupled between the handle and the suture cartridge, a proximal end of the flexible shaft is coupled to a driving mechanism in the handle and a distal end of the flexible shaft is operatively coupled to the belt for driving the needle.

3. The system as claimed in claim 2, wherein a proximal end of the suture cartridge comprises a circular cross section with a plurality of lugs circumferentially arranged on an outer surface of the circular cross section for engagement with corresponding groves provided on an inner surface of the shaft assembly or on an inner surface of an opening at the distal end of the handle for detachable coupling of the suture cartridge with any of the distal end of the shaft assembly and the distal end of the handle.

4. The system as claimed in claim 3, wherein the proximal end of the outer casing of the shaft assembly comprises a plurality of circumferentially arranged lugs that are similar to the a plurality of circumferentially arranged lugs on the proximal end of the suture cartridge, such that it is possible to detachably couple any of the suture cartridge or the proximal end of the shaft assembly to the distal end of the handle.

5. The system as claimed in claim 3, wherein the shaft assembly comprises an articulation joint coupled to the outer casing at the distal end of the shaft assembly to enable pivotally moving the suture cartridge about two mutually perpendicular axes, each perpendicular to the longitudinal axis of the suture cartridge, thereby providing rotational freedom to the suture cartridge during wound closure.

6. The system as claimed in claim 5, wherein the corresponding groves for engaging with the plurality of lugs of the suture cartridge are provided on an inner surface of a yoke of the articulation joint.

7. The system as claimed in claim 6, wherein the handle comprises controls for actuating the driving mechanism and for manipulating the articulation joint.

8. The system as claimed in claim 2, wherein the a flexible shaft is fully flexible along a complete length or comprises two or more shafts coupled to each other through any of: one or more of springs and articulation joints, to provide flexibility to the flexible shaft.

9. The system as claimed in claim 8, wherein the a flexible shaft comprises a pair of couplers and a pair of coupling shafts, each coupling shaft coupled to the two or more shafts at the proximal and the distal ends of the flexible shaft through the respective couplers, and wherein the coupling shafts are configured to operatively couple with the driving shaft of the suture cartridge and the driving mechanism, when the shaft assembly is coupled between the suture cartridge and the handle.

10. The system as claimed in claim 1, wherein the system for wound closure is any of a robotic system, a hand held system and a minimal invasive surgical system.

11. A suture cartridge for extracorporeal and intracorporeal wound closure, wherein the suture cartridge is detachably coupled either to a distal end of a handle of a system for wound closure or to a distal end of a shaft assembly that is coupled to the distal end of the handle of the system for wound closure; wherein, on being coupled to any of the shaft assembly or the handle of the system for wound closure, a driving pulley of the suture cartridge gets operatively coupled to a driving mechanism in the handle to enable the driving mechanism to drive a belt of the suture cartridge to drive a suturing needle positioned in the suture cartridge; and
wherein coupling of the suture cartridge to the handle through the shaft assembly enables intracorporeal wound closure; and coupling of the suture cartridge directly to the distal end of the handle enables extracorporeal wound closure.

12. The suture cartridge as claimed in claim 11, wherein the suture cartridge comprises:
a needle track configured on an outer periphery of a raised projection with the suturing needle positioned in the needle track for movement along the needle track;
a belt configured around the outer periphery of the raised projection such that the belt presses against the suturing needle to move the suturing needle along the needle track as the belt moves;
a pair of idler pulleys to route the belt around the outer periphery of the raised projection; and
a driving pulley to drive the belt;
wherein, when the suture cartridge is coupled to the distal end of the shaft assembly or the handle, the driving pulley gets operatively coupled to a driving mechanism in the handle, either through the shaft assembly, or directly, to enable the driving mechanism for driving the belt and thereby to drive the suturing needle.

13. The suture cartridge as claimed in claim 12, wherein the suture cartridge comprises a base plate and a cover, wherein the raised projection is configured on a distal side of the base plate, and the pair of idler pulleys and the driving pulley are fixed to the base plate.

14. The system as claimed in claim 13, wherein the suture cartridge comprises a driven bevel gear fixedly coupled to the driving pulley, and a driving bevel gear in mesh with the driven bevel gear; and wherein the driving bevel gear is fixed to a distal end of a driven shaft that is configured along a longitudinal axis of the suture cartridge such that when the suture cartridge is coupled to the distal end of the shaft assembly, or the handle, a proximal end of the driven shaft is coupled to a distal end of a flexible shaft of the shaft assembly or directly to the driving mechanism for transferring rotational motion to the belt.

15. The suture cartridge as claimed in claim 14, wherein the driven shaft is rotatably fixed in the longitudinal direction at the proximal end of the base plate such that the driving gear fixed to the driven shaft is in mesh with the driven bevel gear.

16. The suture cartridge as claimed in claim 15, wherein the proximal end of the base plate includes a circular cross section with a plurality of lugs circumferentially arranged on an outer surface of the circular cross section for engagement with corresponding groves provided on an inner surface of an outer casing of the shaft assembly or on an inner surface of an opening at the distal end of the handle for detachable coupling of the suture cartridge with any of the distal end of the shaft assembly and the distal end of the handle.

17. The suture cartridge as claimed in claim 11, wherein the needle track is shaped as a circular arc of more than half circle.

18. The suture cartridge as claimed in claim 17, wherein the suture needle is a circular needle with an arc angle of more than 200 degrees.

19. The suture cartridge as claimed in claim 11, wherein the belt comprises a grove on an outer surface of the belt, the grove being of semi-circular cross section to accommodate the suture needle and simultaneously press the suture needle for movement of the suture needle in the needle track.

20. The suture cartridge as claimed in claim 11, wherein the suture cartridge is configured for use with any of a robotic surgery system, a hand held surgery system and a minimal invasive surgical system.

21. A suture head comprising:
a needle track with the suturing needle positioned in the needle track for movement along the needle track;
a belt configured around the outer periphery of the needle track such that the belt presses against the suturing needle to move the suturing needle along the needle track as the belt moves;
a pair of idler pulleys to route the belt around the needle track; and
a driving pulley to drive the belt;
wherein, operatively coupling the suture cartridge to a driving mechanism of a surgical device enables driving the belt and thereby the suturing needle for suturing.
